(19) United States
(12) Patent Application Publication
(10) Pub. No.: US 2008/0103509 A1
(43) Pub. Date: May 1, 2008

(54) INTEGRATED MEDICAL TRACKING SYSTEM

(76) Inventor: Gunter Goldbach, Worth (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 11/923,694

(22) Filed: Oct. 25, 2007

Related U.S. Application Data
(60) Provisional application No. 60/864,174, filed on Nov. 3, 2006.

(30) Foreign Application Priority Data
Oct. 26, 2006 (EP) 06022395

(51) Int. Cl. A61B 19/00

(52) U.S. Cl. 606/130

(57) ABSTRACT

A medical tracking system includes a tracking unit including an image recording and image signal processing system, and a programmable computer platform integrated with the tracking unit. The programmable computer platform includes a navigation unit configured to implement at least part of medical navigation.

### RELATED APPLICATION DATA

This application claims priority of U.S. Provisional Application No. 60/864,174 filed on Nov. 3, 2006, which is incorporated herein by reference in its entirety.

### FIELD OF THE INVENTION

The present invention relates medical tracking and, more particularly, to an integrated medical tracking system.

### BACKGROUND OF THE INVENTION

Conventional medical tracking systems mainly comprise a tracking unit that includes an image recording and image signal processing system. This technology is based on a hardware that includes digital signal processors (DSPs) and so-called field-programmable gate arrays (FPGAs). The output signal of such conventional tracking systems merely provides coordinates of identified points, for example markers; no other signal processing is performed. Such conventional tracking systems are for example known from U.S. Pat. No. 5,817,105 and U.S. Pat. No. 5,828,770.

FIG. 2 shows a conventional tracking system 11 that includes a data detection unit. The data detection unit typically comprises two or more sensor arrays 2A and 2B (e.g., cameras 2A and 2B) for detecting information concerning the spatial position of tracked/navigated objects (markers) and/or instruments. The cameras 2A and 2B may be rigidly coupled to one another via bar 7.

The tracking system 11 can include image processing means, such as the DSPs and FPGAs noted above, and a calibration value storage means. Broadly speaking, this tracking system 11 provides the data for further processing on a remote computer unit 13. This computer unit 13 can implement further data processing, application-specific programming and data storage. Functions such as graphical user interface programming and interfaces for other devices such as, for example, microscopes, C-arms, etc., may be realized in the computer unit 13. The tracked objects and instruments may be visualized on a screen 14, which can also have input and control functions. FIG. 2 also shows an instrument 5 that is tracked and/or navigated with the aid of a reference marker adaptor 8 coupled to the instrument 5.

One problem with such conventional systems is the relatively complicated setup procedures (e.g., the devices can be relatively complicated to handle and to connect to each other). Further, such systems comprise a number of different devices and, thus, can require significant space.

### SUMMARY OF THE INVENTION

A programmable computer platform is provided in a tracking system, e.g., one that is integrated with a tracking unit of the tracking system. In other words, a relatively unintelligent coordinate detection machine is turned into an intelligent, integral unit. With the aid of the programmable computer platform, functions within the framework of medical navigation that were previously performed outside the tracking system can be integrated into the tracking system. Further, it is particularly advantageous that the miniaturization of computer platforms is relatively far advanced, and that by incorporating these features into a tracking system, integrated hardware solutions for tracking can be provided that exhibit a very small overall size and, therefore, are also easy to handle.

Where tracking systems are mentioned herein, reference is often made to optical tracking systems, which are of course within the scope of the present invention. However, the present invention also is applicable to other types of medical tracking systems, such as, for example, magnetic tracking systems or the like.

It is possible to equip the programmable computer platform with a particular function, several functions or a large number of functions that previously were performed by external navigation hardware. While even a few transferred features make the tracking system itself more versatile, transferring a large number of tasks can result in the complete elimination of an additional data processing and storage unit. Any degree of transfer may have great advantages for certain applications; the medical tracking system in accordance with the present invention provides the option of performing such functions, which conventionally have been implemented externally, directly in the tracking system. Other advantages include significantly improved ergonomic handling and significantly improved setup options, the elimination of a number of external cable connections, the option of providing a stable system casing for the tracking system as a whole, and the optimization of maintenance and service features.

The tracking system can be realized as an assembly that includes the tracking unit and the computer platform. The tracking unit and the computer platform can be accommodated in a casing, in particular in a single casing. Further, it is possible for the programmable computer platform to include at least a part of a medical navigation unit. In other words, navigation tasks can be transferred to the computer platform integrated within the tracking system.

A multi-purpose broadband interface can be assigned to the tracking system, in particular to the computer platform or navigation unit, namely for data transfer with instruments or devices that are involved in tracking. Such instruments or devices can be microscopes, C-arm x-ray devices or similar devices in a medical environment that are also detected during medical navigation. Additionally, a mass storage device, in particular an exchangeable mass storage device, can be assigned to the tracking system, in particular to the computer platform or navigation unit. The mass storage device can be a hard drive, for example, or writable a data medium such as CDs, DVDs or large chip memories.

Within the framework of the invention, it is possible for a remote control device to be assigned to the tracking system, in particular to the computer platform or navigation unit. A graphical user interface or display means, which can be connected to the tracking system, in particular detachably via cables or via a wireless connection, can be assigned to the tracking system or to one of the components described above. Other means that can be assigned to the tracking system include, for example, an illumination means for the operating field, such as a marker illumination means, or a control interface for the control means for controlling the tracking system.

As already described above, it is possible to assign individual functions or a number of functions to the computer platform or navigation unit. These functions, for example, can include:
a synchronous image acquisition function;
an image processing function;
a marker identification and/or tracking function;
an instrument identification and/or tracking function;
a storage function, such as for application-specific information, patient information, updatable tracking system operational information (updates) or information concerning medical apparatus, instruments or devices used;
a remote login function;
a function for graphically visualizing and/or graphically superimposing instruments or instrument shapes;
a function for implementing or performing or assisting application-specific tasks.

Combining image acquisition, image processing, a human-machine interface and visualization means, on the basis of a programmable computer platform, is specifically advantageous for stereoscopic camera setups in which it is important for images from two or more data detection units to be simultaneously processed.

Individual and separate and/or independently existent aspects of the present invention include:
a tracking system comprising an integrated programmable computer platform;
a tracking system comprising an integrated multi-purpose broadband interface that is not limited to point-to-point connections;
a tracking system comprising an integrated mass storage device;
a tracking system comprising a remote control feature.

The major technical changes as compared to conventional systems are that the integrated tracking systems allow advanced image processing and communication by integrating a complete programmable computer platform comprising mass storage devices, an integrated RAM and generic (high-speed) interfaces for connecting other external devices, as preparation for remote control, service tasks and update functions. User-specific software may be downloaded to the tracking system using the multi-purpose interface. The option of providing a small size and low weight for the integrated unit allows new setup options and eliminates the need for an additional computer to control and process the sensor data information. A number of cable connections can be eliminated, and the display and control devices can be flexibly embodied and software-configured to control the integrated tracking system. Remote control for the integrated tracking system is possible with the aid of very small and uncomplicated devices such as for example PDAs, laptops or also, for example, mobile phones and/or other terminal devices.

### BRIEF DESCRIPTION OF THE DRAWINGS

The forgoing and other features of the invention are herein after discussed with reference to the drawing.

FIG. 1 is a schematic representation of an exemplary tracking system in accordance with the invention.

FIG. 2 is a schematic representation of a conventional tracking system.

### DETAILED DESCRIPTION

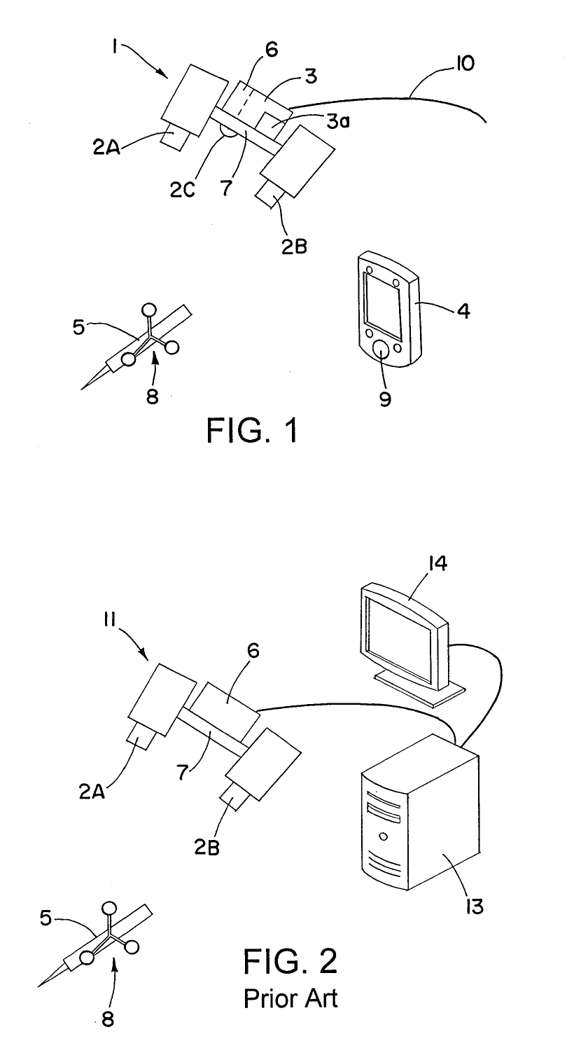

FIG. 1 illustrates an exemplary tracking system 1 in accordance with the invention. The tracking system 1 includes two cameras 2A and 2B coupled to one another via bar 7. The cameras 2A and 2B are operative to record stereoscopic images, for example, of an instrument 5 and its reference marker array 8. The tracking system also is operative to perform image processing 6.

In addition, the tracking system 1 also includes a programmable computer platform 3 (e.g., a computer system) wherein tracking functional units, including programming and a mass storage device 3a, are completely integrated in one unit. Additionally, the tracking system 1 of FIG. 1 includes a generic interface 10 having a large band width that can serve as a single and/or multi-purpose interface. Therefore, complicated wiring is unnecessary. Remote devices such as, for example, a display device 4 and other medical equipment such as C-arms, microscopes, etc. can be connected to the tracking system 1 with the aid of this interface 10.

The interface 10 also serves to connect the integrated tracking system 1 directly to a data storage and database network such as, for example, a gigabit Ethernet interface, a W-LAN interface or a high-speed fibre optic interface. Another specific feature of this interface is that it no longer need be a point-to-point connection to a host computer, but rather allows a number of devices to be connected via a physical interface unit.

Depending on the type of the sensor system 2A, 2B, the tracking system 1 can include illumination means 2C. The illumination means 2C can be, for example, an infrared illumination means for markers, such as markers on the reference array 8 of the instrument 5. Such illumination means on the tracking system are typically used on systems with wireless instruments.

Even a small device 4, such as a graphic user interface and system controller 4, turns the tracking system 1 into a usable medical navigation system. The device 4 includes a small screen (graphic output) and, via a W-LAN connection, for example, fulfils a number of image output and control functions for the integrated tracking system 1. Possible implementations for the device 4 for controlling the tracking system 1 include, for example, a PDA (handheld computer), a tablet PC, a laptop PC, a remote terminal unit comprising a display and a keyboard, or in particular cases possibly even a mobile phone. The device 4 can include various input devices such as, for example, a touch screen, buttons 9, microphones for speech control, a motion detector or other input means. The device 4 can comprise a software programming feature, or can also be a simple pre-configured control device.

The tracking system 1 of FIG. 1 simplifies the overall setup, reduces the number of components needed for medical tracking and navigation, and provides a completely integrated tracking system in a single casing. It thus makes medical tracking and medical navigation flexible and simple.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A medical tracking system, comprising:
a tracking unit including an image recording and image signal processing system, and
a programmable computer platform integrated with the tracking unit, wherein the programmable computer platform includes at least a part of a medical navigation unit.

2. The tracking system according to claim 1, wherein the image recording and image signal processing system and the programmable computer platform are formed as a single assembly.

3. The tracking system according to claim 1, wherein the tracking unit and the programmable computer platform are arranged in a common casing.

4. The tracking system according to claim 1, further comprising a multi-purpose broadband interface for data transfer with instruments or devices utilized during navigation.

5. The tracking system according to claim 1, further comprising a mass storage device.

6. The tracking system according to claim 5, wherein the mass storage device is a removable mass storage device.

7. The tracking system according to claim 5, wherein the mass storage device is a hard drive.

8. The tracking system according to claim 1, further comprising a remote control device communicatively coupled to the tracking system, said remote control device operative to provide a user interface for the tracking unit and/or the programmable computer platform.

9. The tracking system according to claim 1, further comprising a graphical user interface or display device coupled to the tracking system.

10. The tracking system according to claim 11, wherein the graphical user interface and/or display device are communicatively coupled to the tracking system via a wireless connection.

11. The tracking system according to claim 1, comprising at least one of:
a marker illumination device for the operating field; or
a control interface for controlling the tracking system.

12. The tracking system according to claim 1, wherein the programmable computer platform is configured to perform at least one of:
a synchronous image acquisition function;
an image processing function;
a marker identification and/or tracking function;
an instrument identification and/or tracking function;
a storage function;
a remote login function;
a function for graphically visualizing and/or graphically superimposing instruments or instrument shapes;
a function for implementing or performing or assisting application-specific tasks.

13. The tracking system according to claim 12, wherein the storage function includes storage of at least one of application-specific information, patient information, updatable tracking system operational information (updates) or information concerning medical apparatus, instruments or devices.
